(19) United States
(12) Patent Application Publication
(10) Pub. No.: US 2022/0126311 A1
(43) Pub. Date: Apr. 28, 2022

(54) ELECTROSTATIC SPRAY SYSTEM

(71) Applicant: ElectroDefense Corp., Ocala, FL (US)

(72) Inventors: Gabriel Doher, Ocala, FL (US); Chad Doher, Ocala, FL (US); Gregg Murfin, Kempsey (GB); James Brown, Shanghai (CN); Yamile Chamoun, Ocala, FL (US)

(21) Appl. No.: 17/078,083

(22) Filed: Oct. 22, 2020

(51) Int. Cl. B05B 5/035; B05B 5/16; F21V 33/00

(52) U.S. Cl. CPC B05B 5/035; G01S 19/13; F21V 33/0064; B05B 5/1691

(57) ABSTRACT

A spray system for spraying a fluid, the spray system including a tank for holding the fluid; and a sprayer including a fluid conductor, a fluid mover, at least one first nozzle and a second nozzle, the fluid conductor including an inner diameter, a first end configured to be connected to the tank, a second end configured to be selectively connected to one of the at least one first nozzle and a second nozzle, wherein the second nozzle including a through opening at least about the same size as the inner diameter, wherein the fluid mover is configured to draw the fluid from the tank through one of the at least one first nozzle to create a spray of the fluid and the second nozzle to return at least a portion of the fluid to a storage tank.

### BACKGROUND OF THE INVENTION

### 1. The Field of the Invention

The present invention relates to an electrostatic sprayer. More specifically, the present invention is directed to an electrostatic sprayer capable of self-cleaning and capable of returning unused fluid to a storage tank.

### 2. Background Art

The COVID-19 pandemic has changed the manner in which people interact physically in many ways. Physical distancing has become the norm and various requirements and restrictions are imposed on businesses in order for economic activities to take place while lessening the impact of the very communicable and airborne COVID-19 coronavirus. As COVID-19 coronavirus can infect an individual without causing any symptoms, many individuals who are infected are unknowingly passing the disease onto others around them. In addition to physical distancing, health professionals, scientists, health officials, epidemiologist and virologist commonly believe that, at minimum, the use of a face mask reduces the potential for an infected individual from infecting others while providing a physical barrier to reduce transmission by droplets from an infected individual to the face mask wearer. The authorities of many publicly-accessible venues have required the use of face mask for every individual entering the venues as a condition for the venues to remain accessible for fear that they become the grounds for the COVID-19 pandemic to be perpetuated while there are still no effective COVID-19 treatment regime and vaccines to combat COVID-19. While physical distancing and the use of face masks are thought to be effective in combating transmission of COVID-19, the use of disinfectant to reduce the viability of COVID-19 for transmission can further reduce the number of transmissions. Prolonged use of such face masks can cause discomfort or even allergic reactions, causing their wearers to not consistently use the face masks while the situations call for their use out in the publicly accessible venues. Further, in situations where wearers need to temporarily remove their face masks for food and drinks, one of the loops of a face mask is unseated from an ear while the other clings insecurely to the other ear, risking accidental soiling of the face mask or even the loss of the face mask. In some cases, wearers would remove face masks altogether, leaving the wearers to potentially leave COVID-19 virus in the surrounding areas of the wearers. The same measures can be taken to fight other highly transmittable diseases whether airborne or not.

There exists a need for a system capable of applying disinfectant over large areas as COVID-19 and other highly transmittable disease-causing viruses or bacteria are not visible to the naked eye. As large areas are required to be disinfected, the proper application of a disinfectant is critical in ensuring that the disinfectant is truly effective in reducing the disease-causing pathogens. There exists a need for a fluid sprayer that is easy to use, easy and safe to handle at the end of a shift after the fluid sprayer has been used and methodologies to ensure that disinfecting work has been performed as planned.

### SUMMARY OF THE INVENTION

In accordance with the present invention, there is provided a spray system for spraying a first fluid, the spray system including a tank for holding the first fluid; and a sprayer including a fluid conductor, a fluid mover, at least one first nozzle and a second nozzle, the fluid conductor including an inner diameter, a first end configured to be connected to the tank, a second end configured to be selectively connected to one of the at least one first nozzle and a second nozzle, wherein the second nozzle including a through opening at least about the same size as the inner diameter, wherein the fluid mover is configured to draw the first fluid from the tank through one of the at least one first nozzle to create a spray of the first fluid and the second nozzle to return at least a portion of the first fluid to a storage tank.

In one embodiment, the at least one first nozzle is a 40-micron, 3.4 oz. per minute nozzle, an 80-micron, 5.1 oz. per minute nozzle or a 110-micron, 13.4 oz. per minute nozzle. In one embodiment, the fluid mover is a diaphragm pump.

In one embodiment, the sprayer further includes a front end and a rear end, wherein the first fluid is configured to be sprayed from the front end of the sprayer in a direction from the rear end of the sprayer to the front end of the sprayer and the spray system further includes a light source configured to be projected upon an area from the sprayer in the direction, the light source is coupled to the sprayer, wherein the light source illuminates the first fluid sprayed onto the area, enabling coverage of the first fluid in the area to be determined by a viewer having a line of sight of from the rear end of the sprayer to the front end of the sprayer.

In one embodiment, the light source includes a Light Emitting Diode (LED). In one embodiment, the output of the light source ranges from about 500 to about 600 lumens. In one embodiment, the spray system further includes a flow meter configured for providing a flowrate through one of the at least one first nozzle and the second nozzle. In one embodiment, the first fluid is a disinfectant. In one embodiment, the spray system further includes a three-way valve disposed upstream of the fluid mover, the three-way valve including a first input port, a second input port and an output port, the first input port configured to be connected to the tank, the second input port configured to be connected to a second fluid source, the output port configured to be connected to the at least one first nozzle, the passages through the first input port and the second input port are mutually exclusive, wherein when the first input port is active, the first fluid is urged through the output port and the at least one first nozzle by the fluid mover and when the second input port is active, the second fluid source is urged through the fluid mover and the at least one first nozzle by the fluid mover. In one embodiment, the second fluid source is water. In one embodiment, the spray system further includes a controller and a flow meter functionally connected to the controller, the flow meter is configured to measure a flowrate through the at least one first nozzle, wherein the controller is configured to provide a real-time total volume through the at least one first nozzle. In one embodiment, the spray system further includes a controller and a Global Positioning System (GPS) module functionally connected to the controller, the controller configured for reporting a location of the spray system by the GPS module, the location is compared to an expected location, wherein if the location exceeds the expected location by a threshold, a warning is raised by the controller.

In one embodiment, the spray system further includes a controller, a flow meter functionally connected to the controller and a GPS module functionally connected to the controller, the flow meter is configured to measure a flowrate through the at least one first nozzle, the controller is configured to provide a real-time total volume through the at least one first nozzle, the controller is configured for reporting a location of the spray system, the location is compared to an expected location, wherein if the location exceeds the expected location by a threshold and the real-time total volume is being reported, a warning is raised by the controller.

In one embodiment, the spray system further includes a level indicator configured for indicating a level of the first fluid in the tank, wherein when the level of the first fluid is indicated by the level indicator to be lower than a threshold level, the operation of the fluid mover is terminated.

In accordance with the present invention, there is provided a spray system for spraying a fluid onto an area, the spray system including:
(a) a sprayer including a front end and a rear end, wherein the fluid is configured to be sprayed from the front end of the sprayer in a direction from the rear end of the sprayer to the front end of the sprayer; and
(b) a light source configured to be projected upon the area from the sprayer in the direction, the light source is coupled to the sprayer, wherein the light source illuminates the fluid sprayed onto the area, enabling coverage of the fluid in the area to be determined by a viewer having a line of sight of from the rear end of the sprayer to the front end of the sprayer.

In one embodiment, the light source includes a Light Emitting Diode (LED). In one embodiment, the spray system further includes a flow meter configured for providing a flowrate through one of the at least one first nozzle and the second nozzle.

In accordance with the present invention, there is provided a sprayer including: an elongated handheld sprayer including a central axis, a front end, a rear end and a handle extending from a location on the central axis at an incline of about 65 to about 85 degrees to the central axis, the location is defined as a distance from the front end of the handheld sprayer on the handheld sprayer, wherein the distance satisfies a ratio of a first weight to a second weight of about 1.0, the first weight is a weight of the handheld sprayer from the front end of the handheld sprayer to the location, the second weight is a weight of the handheld sprayer from the location to the second end of the handheld sprayer.

In one embodiment, at least of portion of the handheld sprayer between the location and the rear end of the handheld sprayer is configured to be supported on a user's forearm.

An object of the present invention is to provide a fluid sprayer having mechanisms for tracking the work product performed using the fluid sprayer.

Another object of the present invention is to provide a fluid sprayer that is long-lasting.

Another object of the present invention is to provide a fluid sprayer that is easy to handle between shifts.

Whereas there may be many embodiments of the present invention, each embodiment may meet one or more of the foregoing recited objects in any combination. It is not intended that each embodiment will necessarily meet each objective. Thus, having broadly outlined the more important features of the present invention in order that the detailed description thereof may be better understood, and that the present contribution to the art may be better appreciated, there are, of course, additional features of the present invention that will be described herein and will form a part of the subject matter of this specification.

### BRIEF DESCRIPTION OF THE DRAWINGS

In order that the manner in which the above-recited and other advantages and objects of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a diagram depicting a present spray system.

FIG. 2 is a front view of a present spray system.

FIG. 3 is a side view of a present spray system.

FIG. 4 is a top view of a present spray system.

FIG. 5 is a side view of a user using a present spray system.

FIG. 6 is a diagram depicting a manner in which a sprayer of a spray system is being held in a user's hand.

FIG. 7 is a diagram depicting a present spray system where a remaining fluid is returned to a storage tank.

FIG. 8 is a diagram depicting a present spray system where the fluid flow path of the sprayer is being rinsed.

FIG. 9 is a functional diagram of a present spray system.

### PARTS LIST

2 —electrostatic spray system
4—handheld sprayer
6—nozzle selector
8—conductor
10—tank
12—pump
14—shoulder strap
16—spray nozzle
18—return nozzle
20—handle
22—user
24—user's hand
26—user's arm
28—length of front portion
30—length of rear portion
32—light source
34—incline angle between handle and handheld sprayer
36—central axis representing handheld sprayer
38—disinfectant-covered area
40—area not covered by disinfectant
42—light beam
44—storage tank
46—lid
48—height of tank
50—width of tank
52—length of handheld sprayer
54—handle
56—height of tank including handle
58—depth of tank
60—level indicator
62—water source
64—three-way valve
66—Global Positioning System (GPS)
68—flow meter
70—controller
72—internet
74—computing device
76—switch for activating or deactivating cleaning routine
78—switch for activating or deactivating spraying routine
80—switch for activating or deactivating fluid return routine
82—cap
84—waist strap
86—surface, e.g., floor surface
88—spray stream
90—sternum strap
92—spray distance
94—trigger

### Particular Advantages of the Invention

The present spray system includes a fluid return or “dumping” feature which allows the remaining fluid in the tank to be transferred back from the tank to the storage tank before the sprayer is rinsed off via a cleaning feature to rid the spray system of corrosive fluid or disinfectant which the spray system is designed to dispense. This reduces the exposure of parts of the spray system susceptible to corrosion and ensures that the contents of the tank are accounted for to avoid theft, misuse and wastage of the contents of a tank left behind after each spray job.

The present spray system includes a mechanism for reporting and verifying real time tank content use. If a discrepancy has been detected, a job can be flagged and the data associated with the job can be reported to management. For instance, if an application area is expected to require a certain amount of disinfectant and the actually detected amount of the fluid used is much less than the expected amount, the disinfectant is thought to have been inappropriately applied and that the incident is reported and investigated further. If an excessive amount has been applied to an area, e.g., a larger than expected amount of contents have been applied in the area, the incident is reported and investigated further. If an area of application is outside of the expected area of application, the incident is reported and investigated further.

### DETAILED DESCRIPTION OF A PREFERRED EMBODIMENT

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

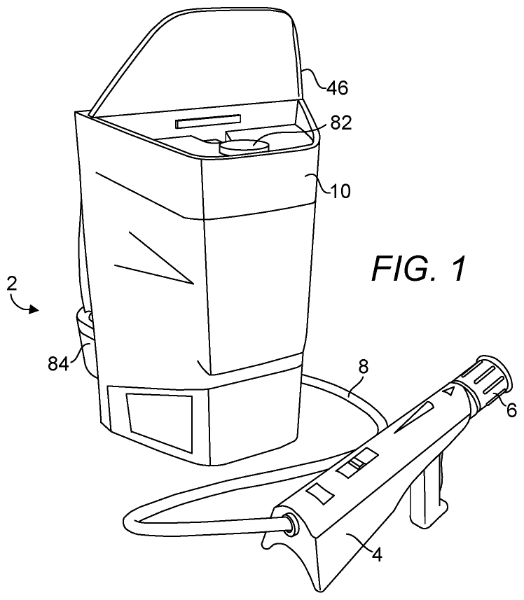

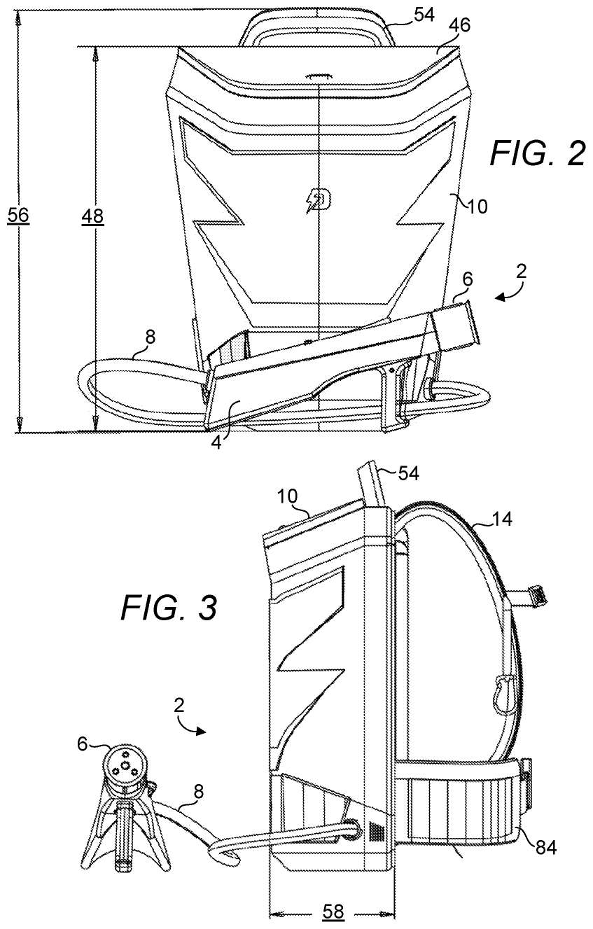

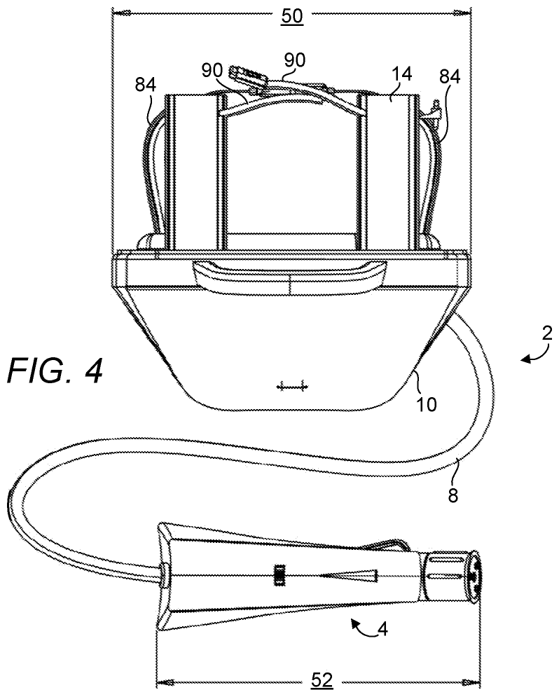

FIG. 1 is a diagram depicting a present backpack-style spray system 2. FIG. 2 is a front view of a present spray system 2. FIG. 3 is a side view of a present spray system 2. FIG. 4 is a top view of a present spray system 2. The present spray system 2 includes a tank 10 for holding a fluid, e.g., about 2.25 Gal., to be sprayed, a handheld sprayer 4 including a nozzle selector 6 where the sprayer 4 receives the fluid to be sprayed from the tank through a conductor 8. In one embodiment, the conductor 8 is a 4-ft Teflon acid/corrosion-resistant hose. Access to the interior of the tank 10 is obtained through an opening that is removably closed using a cap 82. The cap can be removed such that the tank 10 can be filled with a fluid to be sprayed and replaced once the tank 10 has been sufficiently filled. A lid 46 pivotably mounted on top of the tank obscures the cap 82, making it less likely for the cap to be tampered while its user is momentarily away from the spray system 2. A handle 54 extending from a side wall of the tank 10 provides a grasp point for a user of the tank 10 and doubles as a rotational travel limit for the lid 46 while the lid 46 is disposed in its open position. In one embodiment, the tank is constructed from a polymeric material or another high strength per unit weight and corrosion resistant material. In one embodiment, the dry weight and wet weight of the present spray system is about 15 lbs. and about 33 lbs., respectively.

Two shoulder straps 14 extend from the same side wall to facilitate carrying of the tank on its user's shoulders for an ergonomic and comfortable posture for the user. A pair of complementary waist straps 84 are provided to further secure the tank in use and transfer and balance the weight of the tank onto the user's torso. A pair of complementary sternum straps 90 are disposed transversely across the shoulder straps 14 to further allow the user to enhance even distribution of the weight carried by the user.

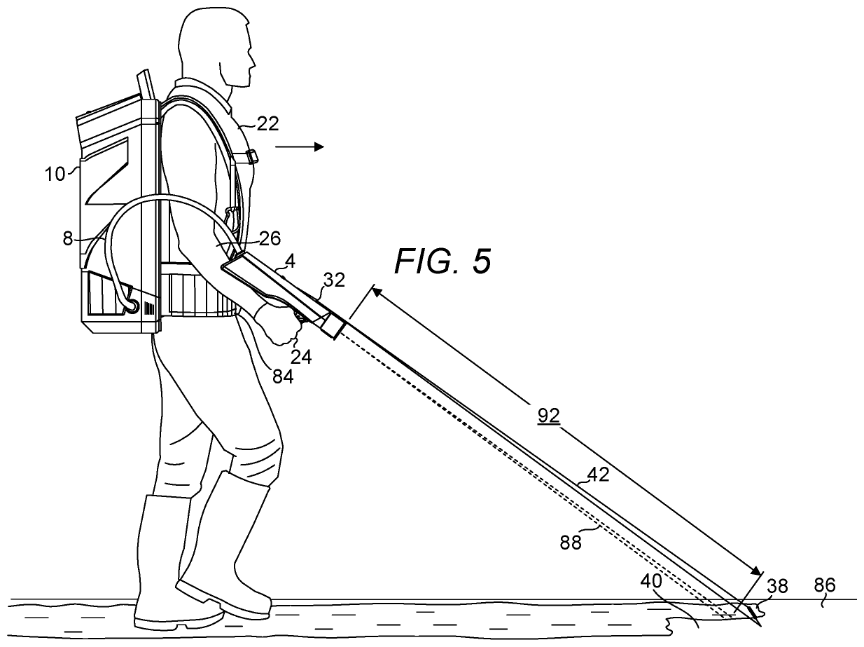

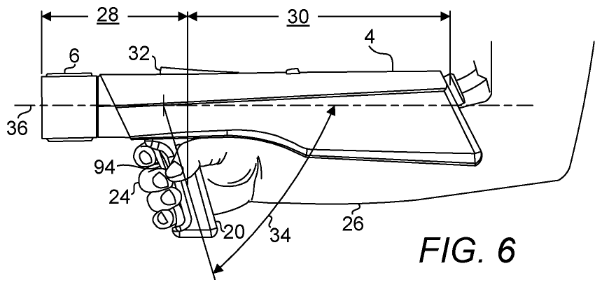

FIG. 5 is a side view of a user 22 using a present spray system. It is shown a spray system 2 being used to apply a spray stream 88, of, e.g., disinfectant, etc., to a surface 86, e.g., floor. It shall be noted that, in use, a portion of the handheld sprayer 4 is supported on the user's arm 26. FIG. 6 is a diagram depicting a manner in which a sprayer of a spray system is being held in a user's hand 24. The sprayer 4 includes a front end and a rear end, wherein the fluid is configured to be sprayed from the front end of the sprayer through a nozzle disposed in the nozzle selector 6, in a direction from the rear end of the sprayer 4 to the front end of the sprayer 4. There is further provided a light source 32 configured to be projected upon an area ahead of the user, in the direction the sprayer is pointed in via its light beam 42, the light source is coupled to the sprayer and disposed on or above an upper portion of the sprayer 4. In use, the light source illuminates, e.g., by reflection, the fluid, e.g., droplets, being sprayed or the fluid sprayed onto the area, enabling coverage of the fluid in the area to be determined by a viewer having a line of sight of from the rear end of the sprayer to the front end of the sprayer. Based on this information, the user can aim the sprayer 4 at an area 40 still to be covered by the fluid and avoid redundant application of the fluid to areas already covered by the fluid. The optimum spray distance 92 ranges from about 3 ft to about 6 ft.

In one embodiment, the light source includes a Light Emitting Diode (LED) as this type of light source sufficiently illuminates a fluid-covered area 38 while consuming less power than other commonly available light sources. In one embodiment, the output of the light source ranges from about 500 to about 600 lumens, i.e., an output sufficient to highlight disinfectant-covered areas and sufficient for a user to distinguish a disinfectant-covered areas from areas not yet covered by disinfectant. In one embodiment, the spray system further includes a flow meter configured for providing a flowrate through one of the at least one first nozzle and the second nozzle.

The sprayer 4 is essentially an elongated handheld sprayer including a central axis, a front end, a rear end and a handle 20 extending from a location on the central axis at an incline 34 of about 65 to about 85 degrees to the central axis 36, the location is defined as a distance from the front end of the handheld sprayer on the handheld sprayer. The distance satisfies a ratio of a first weight to a second weight of about 1.0, the first weight is a weight of the handheld sprayer from the front end of the handheld sprayer to the location, the second weight is a weight of the handheld sprayer from the location to the second end of the handheld sprayer. Applicant discovered that by disposing the weight ratio of the front and rear portions in such a manner, the balanced or even weights render the use of the handheld sprayer less tiring so that the user will have to take less frequent breaks and can complete a task more quickly. Further, by distributing the weight of the handheld sprayer and by providing a gun-style sprayer where the sprayer is secured using a hand 24, the risk of developing carpal tunnel syndrome and stresses on the wrist and arm due to prolonged use of the present spray system is greatly reduced. A trigger 94 is disposed on the handle where the trigger 94 is configured to activate spraying. In one embodiment, the trigger 94 is acts as an on-off switch which turns on all the parts involving in making spraying possible, e.g., a pump for drawing a fluid from the tank 10 and sending the fluid to be sprayed via a nozzle of the nozzle selector 6 so long as the trigger 94 is depressed. Further provided on or in close proximity to the handle 20 is a trigger lock for extended use of the sprayer. In one embodiment, the trigger 94 is functionally coupled with a trigger lock which secures the trigger 94 in an on position such that the user's grasp can be released while a spray continues to occur.

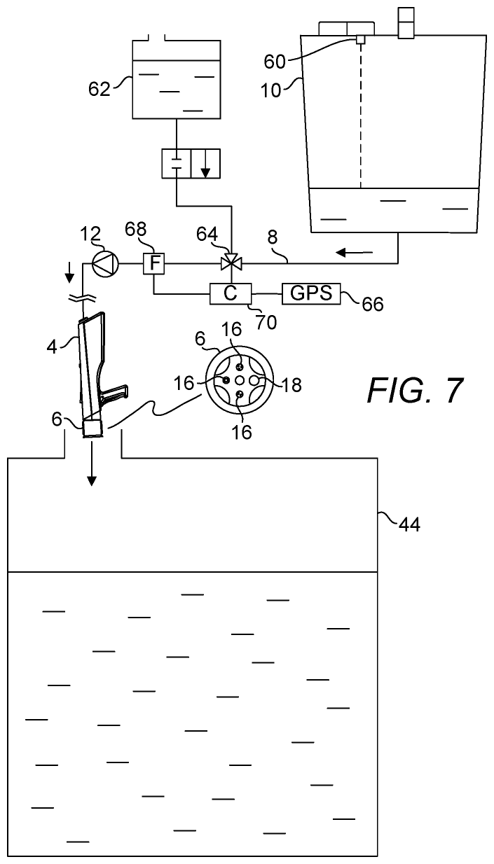

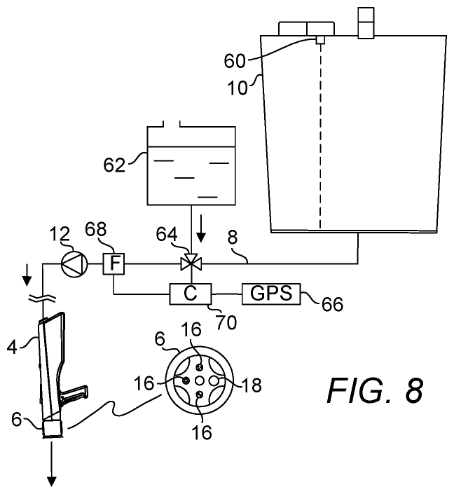

FIG. 7 is a diagram depicting a present spray system where a remaining fluid is returned to a storage tank 44. FIG. 8 is a diagram depicting a present spray system where the fluid flow path of the sprayer is being rinsed. The spray system includes a tank 10 for holding a fluid; and a sprayer including a fluid conductor 8, a fluid mover, e.g., a pump 12, a plurality of spray nozzles 16 and a fluid return nozzle 18. The fluid conductor includes an inner diameter, a first end configured to be connected to the tank 10, a second end configured to be selectively connected to one of the at least one spray nozzle 16 and a fluid return nozzle 18, wherein the fluid return nozzle 18 includes a through opening at least about or substantially the same size as the inner diameter. Note that for the pump 12 to be fluidly connected to both the tank 10 and the second fluid source 62, the pump 12 is disposed at a downstream location from the three-way valve 64. In one embodiment, the second fluid source 62 can be a second tank disposed in close proximity to the tank 10. However, this may not be practical for a user who is striving to transport a minimum weight while applying the contents of the tank 10. In another embodiment, the second fluid source 62 is municipal water and it is connected to the three-way valve 64 only when the cleaning of the sprayer is required. The valve which makes this connection possible is disposed in a normally closed position until cleaning of any one or all the nozzles is desired. The pump 12 is configured to draw the fluid from the tank 10 through one of the at least one spray nozzles 16 to create a spray or a stream that includes the fluid and the fluid return nozzle 18 to return at least a portion of the fluid to a storage tank 44. In one embodiment, the spray nozzles include a 40-micron, 3.4 oz. per minute nozzle, an 80-micron, 5.1 oz. per minute nozzle and a 110-micron, 13.4 oz. per minute nozzle. The nozzle selector 6 is essentially a selector having a rotary disk having three spray nozzles 16 and a fluid return nozzle 18 having an inner diameter that is similar in size to the inner diameter of the conductor 8. In selecting a nozzle for use, the disk is simply rotated until the desired nozzle is disposed over an exhaust of the conductor 8, removing the need for an additional tool to replace the nozzle.

In this embodiment, there is further provided a level indicator 60 configured for indicating a level of the fluid of the tank 10, wherein when the level of the fluid is indicated by the level indicator 60 to be lower than a threshold level, the operation of the pump 12 is terminated, preventing air from entering the system and damaging the pump. A level indicator 60 may include optical level switches, a capacitor level sensor, an ultrasonic sensor, a microwave/radar sensor, a vibrating/tuning fork or a conductivity/resistance float switch, etc. If the operation of the pump 12 is left uncontrolled and the fluid level of the tank 10 drops below a critical level, air can enter the spray system through the tank 10 which will subsequently travel through the conductor 8 to the pump 12, causing the pressure of the fluid in the conductor 8 to drop drastically and reducing the output of fluid from a spray nozzle 16. In one embodiment, the pump 12 is a diaphragm pump capable of self-priming that can help remove trapped air in the conductor 8 if such a condition did occur although the normal operation of the present spray system should prevent it from occurring in the first place. Further, if air does get trapped within the conductor 8, a fluid return function disclosed elsewhere herein can also be used to expel the trapped air. In one embodiment, a tank full of fluid may be applied to cover over about 25,000 sq. ft of surfaces. In one embodiment, the level indicator further provides indications as to whether the fluid level of the tank is “full,” “middle,” “low” or “empty.” A “full” level indicates that no fluid shall be added to the tank 10. A “middle” level indicates that some fluid of the tank 10 has been used but otherwise there is nearly sufficient fluid left in the tank 10 to carry out a spraying task where a full tank is required. A “low” level indicates that the tank 10 should be refilled before the next spraying task is carried out, especially if the user will need to travel great distances to carry out the spraying task. An “empty” status indicates that the tank 10 must be refilled for the next spraying task.

In one embodiment, the present spray system further includes a flow meter 68 configured for providing a flowrate through one of the spray nozzles 16 and the fluid return nozzle 18. In one embodiment, the first fluid is a disinfectant. In one embodiment, the spray system further includes a three-way valve 64 disposed upstream of the pump 12, the three-way valve 64 including a first input port, a second input port and an output port, the first input port configured to be connected to the tank 10, the second input port configured to be connected to a second fluid source 62, the output port configured to be connected to the at least one spray nozzle 16, the passages through the first input port and the second input port are mutually exclusive. In other words, when the first input port is active, only the first fluid is urged through the output port and at a spray nozzle by the pump 12. When the second input port is active, only the second fluid source is urged through the pump 12 and a spray nozzle 16 by the pump 12. In one embodiment, the second fluid source is water. By running water through a nozzle, the nozzle is rid of residual corrosive disinfectant that comes in contact with the various components of the nozzle, prolonging the life of the nozzle.

In one embodiment, the spray system further includes a controller 70 and the flow meter 68 is functionally connected to the controller 70, the flow meter 68 is configured to measure a flowrate through a spray nozzle 16, wherein the controller 70 is configured to provide a real-time total volume through the spray nozzle 16.

In one embodiment, the spray system includes a controller 70 and a Global Positioning System (GPS) 66 module functionally connected to the controller 70, the controller 70 configured for reporting a location of the spray system by the GPS 66 module, the location is compared to an expected location. If the location differs from the expected location by a threshold distance, e.g., 50 ft, a warning is raised by the controller 70. Armed with a present spray system, the location at which it is used can therefore be verified, reducing the potential that the spray system can be misused. For instance, if the GPS 66 module of a spray system indicates that it is not located at an expected location at a particular time, the spray system is considered to be about to be misused.

In one embodiment, the spray system further includes a controller 70, a flow meter 68 functionally connected to the controller 70 and a GPS 66 module functionally connected to the controller 70, the flow meter 68 is configured to measure a flowrate through a spray nozzle 16, the controller 70 is configured to provide a real-time total volume through the spray nozzle 16, the controller 70 is configured for reporting a location of the spray system, the location is compared to an expected location, wherein if the location differs from the expected location by a distance threshold and the real-time total volume is being reported, a warning is raised by the controller. Here, misuse is not assumed until the spray system has been detected to have been used as indicated by a flow detected in the flow meter 68. Further, the rate at which the contents of a tank 10 is used, e.g., total volume used per unit area, can also be calculated and compared to an expected value. For instance, if the real-time total volume per unit area is much smaller than expected, then it can be inferred than the area as indicated by the GPS data has been insufficiently sprayed. Conversely, if the real-time total volume per unit area is much larger than expected, then the area as indicated by the GPS data can be said to have received an excessive amount of the sprayed substance. As the flowrate and GPS data is transmitted in real-time, corrections can be made on the fly, reducing the potential that a large insufficiently sprayed area to require respraying.

Referring back to FIGS. 7 and 8, although the second fluid source 62, flow meter 68, pump 12, three-way valve 64, controller 70, GPS 66 module are all shown to be disposed separately from the tank 10, it is conceivable that the one or more components of this list be disposed physically in close proximity to the tank 10, as part of the “backpack” concept. The sprayer 4 is preferably constructed with as few onboard components as possible for minimal weight. Further, in order to further reduce the weight felt in a user's arm, the sprayer 4 may have its own controller capable of wired or wireless communication, e.g., via Bluetooth, with controller 70.

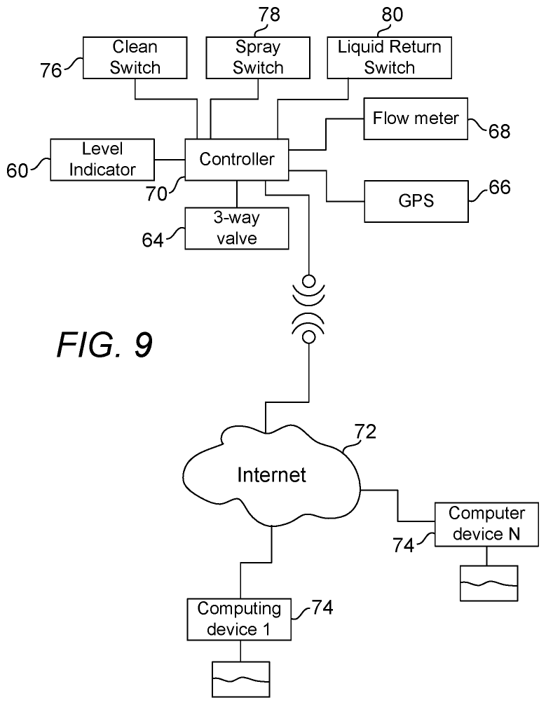

FIG. 9 is a functional diagram of a present spray system. Locally, at the spray system level, controller 70 is functionally connected to a switch 76 used for activating or deactivating a cleaning routine, a switch 78 for activating or deactivating a spraying routine, a switch 80 for activating or deactivating a fluid return routine, a flow meter 68, a GPS 66 module, a three-way valve 64 and a level indicator 60. As the controller 70 is required to communicate real-time data with devices 74, e.g., computing devices, cellular phones, etc., that are located at great distances, controller 70 is configured to communicate with the internet 72 via a wireless communication protocol, e.g., Wi-Fi or cellular protocol. The devices 74 are in turn connected to the internet 72. As real-time data from the spray system is reported to the devices 74, real-time feedback from the supervisors of the devices 74 is possible, making the spraying tasks more effective.

The detailed description refers to the accompanying drawings that show, by way of illustration, specific aspects and embodiments in which the present disclosed embodiments may be practiced. These embodiments are described in sufficient detail to enable those skilled in the art to practice aspects of the present invention. Other embodiments may be utilized, and changes may be made without departing from the scope of the disclosed embodiments. The various embodiments can be combined with one or more other embodiments to form new embodiments. The detailed description is, therefore, not to be taken in a limiting sense, and the scope of the present invention is defined only by the appended claims, with the full scope of equivalents to which they may be entitled. It will be appreciated by those of ordinary skill in the art that any arrangement that is calculated to achieve the same purpose may be substituted for the specific embodiments shown. This application is intended to cover any adaptations or variations of embodiments of the present invention. It is to be understood that the above description is intended to be illustrative, and not restrictive, and that the phraseology or terminology employed herein is for the purpose of description and not of limitation. Combinations of the above embodiments and other embodiments will be apparent to those of skill in the art upon studying the above description. The scope of the present disclosed embodiments includes any other applications in which embodiments of the above structures and fabrication methods are used. The scope of the embodiments should be determined with reference to the appended claims, along with the full scope of equivalents to which such claims are entitled.

What is claimed herein is:

1. A spray system for spraying a first fluid, said spray system comprising:
(a) a tank for holding the first fluid; and
(b) a sprayer comprising a fluid conductor, a fluid mover, at least one first nozzle and a second nozzle, said fluid conductor comprising an inner diameter, a first end configured to be connected to said tank, a second end configured to be selectively connected to one of said at least one first nozzle and a second nozzle, wherein said second nozzle comprising a through opening at least about the same size as said inner diameter,
wherein said fluid mover is configured to draw the first fluid from said tank through one of said at least one first nozzle to create a spray of the first fluid and said second nozzle to return at least a portion of the first fluid to said tank.

2. The spray system of claim 1, wherein said at least one first nozzle comprises a nozzle selected from the group consisting of a 40-micron, 3.4 oz. per minute nozzle, an 80-micron, 5.1 oz. per minute nozzle and a 110-micron, 13.4 oz. per minute nozzle.

3. The spray system of claim 1, wherein said fluid mover is a diaphragm pump.

4. The spray system of claim 1, wherein said sprayer further comprises a front end and a rear end, wherein the first fluid is configured to be sprayed from said front end of said sprayer in a direction from said rear end of said sprayer to said front end of said sprayer and said spray system further comprises a light source configured to be projected upon an area from said sprayer in said direction, said light source is coupled to said sprayer, wherein said light source illuminates the first fluid sprayed onto the area, enabling coverage of the first fluid in the area to be determined by a viewer having a line of sight of from said rear end of said sprayer to said front end of said sprayer.

5. The spray system of claim 4, wherein said light source comprises a Light Emitting Diode (LED).

6. The spray system of claim 4, wherein the output of said light source ranges from about 500 to about 600 lumens.

7. The spray system of claim 1, further comprising a flow meter configured for providing a flowrate through one of said at least one first nozzle and said second nozzle.

8. The spray system of claim 1, wherein the first fluid is a disinfectant.

9. The spray system of claim 1, further comprising a three-way valve disposed upstream of said fluid mover, said three-way valve comprising a first input port, a second input port and an output port, said first input port configured to be connected to said tank, said second input port configured to be connected to a second fluid source, said output port configured to be connected to said at least one first nozzle, the passages through said first input port and said second input port are mutually exclusive, wherein when said first input port is active, the first fluid is urged through said output port and said at least one first nozzle by said fluid mover and when said second input port is active, said second fluid source is urged through said fluid mover and said at least one first nozzle by said fluid mover.

10. The spray system of claim 9, wherein said second fluid source is water.

11. The spray system of claim 1, further comprising a controller and a flow meter functionally connected to said controller, said flow meter is configured to measure a flowrate through said at least one first nozzle, wherein said controller is configured to provide a real-time total volume through said at least one first nozzle.

12. The spray system of claim 1, further comprising a controller and a Global Positioning System (GPS) module functionally connected to said controller, said controller configured for reporting a location of said spray system by said GPS module, said location is compared to an expected location, wherein if said location differs from said expected location by a threshold distance, a warning is raised by said controller.

13. The spray system of claim 1, further comprising a controller, a flow meter functionally connected to said controller and a GPS module functionally connected to said controller, said flow meter is configured to measure a flowrate through said at least one first nozzle, said controller is configured to provide a real-time total volume through said at least one first nozzle, said controller is configured for reporting a location of said spray system, said location is compared to an expected location, wherein if said location differs from said expected location by a threshold distance and said real-time total volume is being reported, a warning is raised by said controller.

14. The spray system of claim 1, further comprising a level indicator configured for indicating a level of the first fluid in said tank, wherein when the level of the first fluid is indicated by the level indicator to be lower than a threshold level, the operation of said fluid mover is terminated.

15. A spray system for spraying a fluid onto an area, said spray system comprising:
(a) a sprayer comprising a front end and a rear end, wherein the fluid is configured to be sprayed from said front end of said sprayer in a direction from said rear end of said sprayer to said front end of said sprayer; and
(b) a light source configured to be projected upon the area from said sprayer in said direction, said light source is coupled to said sprayer, wherein said light source illuminates the fluid sprayed onto the area, enabling coverage of the fluid in the area to be determined by a viewer having a line of sight of from said rear end of said sprayer to said front end of said sprayer.

16. The spray system of claim 15, wherein said light source comprises a Light Emitting Diode (LED).

17. The spray system of claim 15, further comprising:
(a) a tank for holding the fluid; and
(b) a sprayer comprising a fluid conductor, a fluid mover, at least one first nozzle and a second nozzle, said fluid conductor comprising an inner diameter, a first end configured to be connected to said tank, a second end configured to be selectively connected to one of said at least one first nozzle and a second nozzle, wherein said second nozzle comprising a through opening at least about the same size as said inner diameter,
wherein said fluid mover is configured to draw the fluid from said tank through one of said at least one first nozzle to create a spray of the fluid and said second nozzle to return at least a portion of the fluid to said tank.

18. The spray system of claim 17, wherein said at least one first nozzle comprises a nozzle selected from the group consisting of a 40-micron, 3.4 oz. per minute nozzle, an 80-micron, 5.1 oz. per minute nozzle and a 110-micron, 13.4 oz. per minute nozzle.

19. The spray system of claim 17, wherein said fluid mover is a diaphragm pump.

20. The spray system of claim 17, further comprising a flow meter configured for providing a flowrate through one of said at least one first nozzle and said second nozzle.
